(19) United States
(12) Patent Application Publication
(10) Pub. No.: US 2009/0123569 A1
(43) Pub. Date: May 14, 2009

(54) Coverage indicating technology for skin sealants using tannates

(76) Inventors: John Gavin MacDonald, Decatur, GA (US); Molly K. Smith, Atlanta, GA (US); Iiona F. Weart, Woodstock, GA (US); Phillip A. Schorr, Atlanta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/983,222

(22) Filed: Nov. 8, 2007

(51) Int. Cl. A61K 33/18; A61K 47/08; A61K 47/32; A61P 17/02; A61P 31/04; A61P 31/12; A61P 31/10; A61P 31/02

(52) U.S. Cl. 424/667; 514/772; 514/772.6

(57) ABSTRACT

A composition having various color changing tannates may be used to indicate that the composition has dried. The tannates change color in contact with an acid. The composition may be, for example, a skin prep, skin sealant, food product, paint or other building material or other product that undergoes a phase change. The tannate may be added either directly to the composition, incorporated into a sponge on an applicator through which the composition is dispensed and applied, applied separately or applied simultaneously from a separate reservoir. The amount of tannate in the composition can be adjusted to provide a visual cue to the user of the application area and the extent of cure.

### BACKGROUND OF THE INVENTION

Surgical site infections (SSI) occur following about 2-3 percent of surgeries in the United States with an estimated 500,000 incidents of SSI occurring annually, which can lead to significant patient morbidity and mortality. In addition to the negative impact of such infections on patient health, these potentially avoidable infections contribute significantly to the financial burden experienced by the health care system. SSIs result when an incision becomes contaminated by bacteria, and for most surgeries the primary source of these infection-causing microorganisms is the skin (an exception being surgeries in which the gastrointestinal tract is penetrated).

Various compositions are used to prepare the skin prior to surgery. Skin preparations or “preps” are used to remove some level of microbial load on the skin prior to making an incision. Skin sealant materials are used to protect patients from bacterial infections associated with surgical site incisions and insertion of intravenous needles. Skin preps are applied to the skin and allowed to dry to maximize effectiveness for reducing microorganisms. After the skin prep has dried, the sealant may be applied directly to the skin in liquid form. The sealant forms a coherent film with strong adhesion to the skin through various techniques based on the chemistry of the sealant composition.

Skin preps currently are predominantly povidone-iodine or chlorhexidine gluconate based formulations and may contain alcohol for fast drying and more effective killing of organisms. Time constraints in the operating room and the lack of an indicator that the prep has dried often result in the skin remaining wet when draping and/or surgery begin, creating the possibility of infection. The lack of an indicator can also negatively impact infection since the users cannot know with certainty where the prep and sealant have been applied.

Skin sealants now use a polymer composition that dries to form a film through evaporation of a solvent, for example. Other skin sealants contain monomeric units that polymerize in situ to from a polymeric film. Cyanoacrylate sealants containing alkyl cyanoacrylate monomer are an example of the latter type wherein the monomer polymerizes in the presence of a polar species such as hydroxide, water or protein molecules to form an acrylic film. The resulting film formed serves to immobilize bacterial flora found on the skin and prevents their migration into an incision made during a surgical procedure or skin puncture associated with insertion of an intravenous needle.

Skin sealants may contain additives such as plasticizing agents to improve film flexibility and conformance, viscosity modifiers to aid in application of the liquid composition, free radical and anionic scavengers to stabilize the product prior to use, biocidal agents to kill immobilized bacteria under the film, and the like.

Skin sealants have also been formulated with colorants to help the user apply the liquid composition uniformly to the skin, especially when large areas are to be covered. There are several problems, however, with existing colorants; addition of a colorant directly to the liquid skin sealant composition can negatively impact both in situ polymerization rates and the conversion reaction, in the case of cyanoacrylate compositions, or evaporation rates and the coalescence process in the case of polymer solution compositions. In addition, known colorants do not provide a visual cue to indicate curing of the composition has been completed. Lastly, after completion of the surgical procedure, the colorant in the sealant can obscure the wound site, making it difficult to detect redness associated with surgical site infections, bruising or leakage.

It is clear that there exists a need for a colorant that provides a visual cue to indicate coverage area and/or curing and that does not obscure the wound site.

### SUMMARY OF THE INVENTION

In response to the foregoing difficulties encountered by those of skill in the art, we have discovered that compositions including color changing tannates may be used to indicate that the composition has dried and the area of coverage. Iron tannate changes color in response to an acidic environment. The iron tannate may be added to a skin prep, for example, and the color discharged by a slightly acidic coating like a skin sealant. The tannate may be added either directly to a composition, incorporated into a sponge on the applicator through which the composition is dispensed and applied, applied separately or applied simultaneously from a separate reservoir. The amount of tannate in the composition can be adjusted to provide a visual cue to the user of the application area and the extent of cure.

### DETAILED DESCRIPTION OF THE INVENTION

Tannic acid occurs in the bark and fruit of many plants, notably the bark of the oak species, in sumac and myrobalan. Commercial uses include sizing paper and silks, clarifying beer and wine. Medical uses include external use as an astringent for burns and internally as an astringent and as a heavy metal antidote.

Skin preparations or “preps” are used to remove some level of microbial load on the skin prior to making an incision. Skin preps are applied to the skin and allowed to dry to maximize effectiveness for reducing microorganisms. Skin preps currently are predominantly povidone-iodine or chlorhexidine, gluconate based formulations and may contain alcohol for fast drying and more effective killing of organisms. Povidone iodine, available commercially as Betadine® is estimated to be used in 80 percent of surgeries as a skin preparation. Betadine® skin prep is an aqueous solution of 10 percent povidone iodine having 1 percent titratable iodine content. When Betadine® skin prep is applied to the skin, it imparts and orange-brown color.

Skin sealant materials are used to protect patients from bacterial infections associated with surgical site incisions and insertion of intravenous needles. Skin sealants are often applied directly over or on top of (Betadine®) skin preps. The sealant forms a coherent film with strong adhesion to the skin through various techniques based on the chemistry of the sealant composition. The skin sealants used herein contain a film former and a plasticizer and other optional ingredients like viscosity modifiers to aid in application of the liquid composition, free radical and anionic scavengers to stabilize the product prior to use, biocidal agents to kill immobilized bacteria under the film, and the like.

One film former available in a skin sealant composition is commercially known as InteguSeal® and is available from Medlogic Global, Ltd of Plymouth, England. InteguSeal® skin sealant contains medical grade n-butyl cyanoacrylate monomer (80% w/w). Medical grade cyanoacrylate is double distilled. Non-medical grade cyanoacrylate, in contrast, is single distilled and is typically marketed as a “super glue” type adhesive for gluing a wide variety of substrates together.

It would be useful to medical personnel to know exactly where the skin sealant and prep were applied so that they could be sure that the appropriate area was covered. The inventors believe that providing a skin sealant and/or skin prep which will change color as it dries will provide valuable information for the medical professional. The authors identified the vivid color change potential of tannic acid through laboratory research. The invention discussed herein uses tannic acid and metal salts to give a vivid color which is discharged by the acid nature of the skin sealant. Several variations have been developed using this methodology:

1) Iron tannate in the skin prep with the color discharged by the application of the skin sealant (deep blue to colorless)

2) Tannic acid in the sealant and iron salts on the applicator sponge or in a separate vial within the applicator so that the tannic acid and iron salt react to form iron tannate as the sealant is applied to the skin and the iron tannate has its color discharged by the skin sealant and;

3) Iron salt in the skin prep and tannic acid in the skin sealant to give a timed color development and color discharge during the application process.

The amount of iron tannate in the skin prep or sealant should be between about 0.09 and 10 weight percent. This may be calculated by one skilled in the art based upon the volume of the skin prep or sealant to be used. It should be noted that the term “ppm” or parts per million as used herein denotes one particle of a given substance for every 999,999 other particles. This is roughly equivalent to one drop of ink in a 150 liter (40 gallon) drum of water, or one second per 280 hours (11 days, 16 hours). One part in 10⁶—a precision of 0.0001%.

Tannic add of use in commerce occurs in the bark and fruit of many plants, notably in the bark of the oak species, in sumac and myrobalan. It is produced from Turkish or Chinese nutgall, the former containing 50-60%, the later about 70% tannic acid. The chemistry of tannins is quite complex and non-uniform. Tannins may be divided into two groups: (a) derivatives of flavinols, so-called condensed tannins and (b) hydrolysable tannins (the most important group) which are esters of a sugar, usually glucose, with one or more trihydroxybenzenecarboxylic acids. Tannic add is used for clarifying beer and wine and also as an astringent. It has also been used internally for treatment of diarrhea.

The intensity or brightness of light is expressed in lux (lx), for example, an over cast summer day is estimated to between 30,000 lx and 40,000 lx and a mid-winter day is estimated to be about 10,000 lx. The British Standards Institution Code of Practice for Day-lighting, BS 8206 Part 1 deals in general terms with the code of practice for artificial light. The following gives some general guidance for the light requirements for the work place.

General office, laboratories, kitchen—500 lx

Drawing offices—750 lx

Tool rooms and paintwork—1000 lx

Inspection of graphic reproduction—1500 lx.

Accordingly, for purposes of the present invention “normal light conditions” refers to light conditions of between about 500 lx and 2000 lx, more desirably, from about 750 lx to about 1500 lx as determined in accordance with BS 8206 part 1.

As noted above, there a number of ways to use the color change components with a skin prep/skin sealant system: it may be mixed with the skin sealant, it may be impregnated onto a sponge or wipe which is used to apply the sealant, it may be applied separately from a separate reservoir and it may be applied simultaneously from a separate reservoir in a manner similar to the application of an epoxy.

The application of a tannate to a carrier may be done by the “dip and squeeze” method, known to those skilled in the art. In this method, the carrier (e.g., sponge, nonwoven fabric (wipe), cotton ball or other) is placed in a bath of the tannate and allowed to absorb the tannate. After absorbing the tannate, the carrier is squeezed between, for example, a pair of rollers, to force out excess tannate.

Another method to apply tannate to a carrier is to spray the tannate onto the carrier. Spraying generally does not penetrate the carrier with tannate as well as the dip and squeeze method, though it is generally faster and simpler.

Yet another method to apply a tannate to, for example, a stack of wipes in a storage box, is to add the tannate to the box with the wipes. U.S. Pat. Nos. 4,775,582 and 4,853,281, commonly assigned and incorporated by reference in their entirety. These patents concern a method of maintaining relatively uniform moisture in a stack of wipes. The wipes may be made from polyolefinic microfibers that have been extruded and gathered like spunbond or meltblown fibers, or a combination of both. Common materials for construction of wipers include spunbond and meltblown fibers and fabrics in various arrangements.

The term “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface. Laminates of spunbond and meltblown fibers may be made, for example, by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step.

Such fabrics usually have a basis weight of from about 0.1 to 12 osy (6 to 400 gsm), or more particularly from about 0.75 to about 3 osy. Multilayer laminates may also have various numbers of meltblown (abbreviated as “M”) layers or multiple spunbond (abbreviated as “S”) layers in many different configurations and may include other materials like films (abbreviated as “F”) or coform materials (see U.S. Pat. No. 4,100,324 for descriptions of exemplary “coform” materials), e.g. SMMS, SM, SFS, etc.

Applying the sealant from a separate reservoir may involve the use of dispensers developed for that purpose. One exemplary dispenser has the liquid sealant held in at least one oblong glass ampoule within a rigid nylon housing. The housing has a body and a cap that are slidably connected and it is the cap which holds the ampoule(s). In use, the two parts are moved toward each other to dispense the liquid; the cap moving into the body. Moving the parts together results in breakage of the glass ampoule(s) and dispensing of the liquid. A detent-type locking mechanism holds the body and cap together once they are moved. The locking mechanism consists of slots formed in the cap into which fits a slight protuberance or knoll of plastic formed on the inside surface of the body. Once the ampoule is broken, the liquid travels through a small piece of foam which catches any glass shards that may have been formed by the breakage of the ampoule and thence on to the tip portion of the body. The tip has a number of small holes in it to allow the liquid to pass through. The body tip has a piece of foam on the outside, held in place with a rigid plastic oval-shaped ring that snaps in place on the tip. The outer foam contacts the skin of the patient when the liquid is dispensed. Other types of dispensers may be found in U.S. Pat. Nos. 4,854,760, 4,925,327 and 5,288,159, incorporated herein by reference.

In another embodiment the skin sealant and tannate may be applied separately to the area containing a skin prep. U.S. Pat. No. 5,928,611 describes a dispenser having a skin sealant reservoir and an active ingredient such as a cross linking accelerator or initiator disposed on a foam piece through which the sealant must pass. One could envision the use of such a dispenser having the tannate disposed on the foam piece and the sealant passing though it as it is about to be deposited onto the skin. See also U.S. Pat. No. 6,322,852.

In yet another embodiment, U.S. Pat. No. 6,340,097 describes a dispenser having at least one crushable ampoule within the body of the dispenser which could hold more than one. This would permit one ampoule to hold skin sealant and a second to hold the tannate. When the dispenser was used, it would break both ampoules and the sealant and tannate would mix just before application to the skin.

In addition to being used as a traditional skin sealant, i.e. as a film forming barrier through which a surgical incision is made, the tannate and skin sealant composition may also be used like a bandage to close and/or cover wounds, abrasions, burns, acne, blisters and other disruptions in the skin to protect them from subsequent contamination. The use of the skin sealant composition would therefore not be limited to medical personnel.

Wound protection is critical in permitting the healing process to take place. Traditional adhesive bandages and gauze wound dressings have been used by the consumer to treat/dress acute wounds or skin irritations. Such adhesive bandages are generally passive, in that they offer little or no chemical treatment for wound healing. Rather, they primarily serve to exert low levels of pressure on the wound, protect the wound from exposure to the environment, and absorb any exudates, which are produced from the wound site. Such bandages generally include a base layer, which is the layer seen by the consumer following application of the bandage to the wound. Such a layer is typically formed from a polymeric material such as a film, nonwoven web, or combination thereof, and may be perforated in some fashion to allow for flexibility and/or further breathability. This layer often includes a film component, having a top side surface which is seen by the consumer after application of the bandage to the wound site, and a bottom side surface (skin contacting surface). A skin-friendly adhesive is usually placed over the base layer bottom side surface to provide a means for attaching the bandage to the consumer. Alternatively, a separate adhesive tape is used to attach the bandage/wound dressing to the wound site, if the bandage/wound dressing is of the nonadhesive type. In the center of the base layer bottom side surface is traditionally positioned an absorbent pad for absorbing exudates from the wound. Finally, a non-stick perforated film layer is normally positioned over the absorbent pad layer, to provide a barrier between the absorbent pad and the wound itself. This allows the wound fluid to move through the perforated layer without sticking to the wound site. Typically the absorbent pad in such bandage does not include any medicinal components, although comparatively recently, bandage manufacturers have started including antibiotic agents on or within bandages to encourage wound healing.

The skin sealant composition of this invention can replace this seemingly complicated bandage construction with a single liquid treatment that will dry to a flexible coating that protects a wound much like a bandage would. Additionally, medicaments such as antibiotic agents may be blended in effective amounts with the composition to provide additional benefits in the area of microbial inhibition and the promotion of wound healing. The sealant may be applied to provide an effectively thick coating over the surface of the superficial wound, burn or abrasion. Because the to-be-treated wound is superficial and does not extend beyond the dermal layer, any polymeric residues diffusing into or forming in the wound will be naturally extruded from the skin. Generally, the sealant provides an adhesive film coating over the wound area which when set is satisfactorily flexible and adherent to the tissue without premature peeling or cracking. The coating generally has a thickness of less than about 0.5 millimeter (mm).

Sealant coatings of such thicknesses form a physical barrier layer over superficial wounds which provide protection for the wound in the same manner as a conventional bandage. Specifically, the coating provides an almost airtight, waterproof seal around the wound which does not need to be replaced when the wound gets wet. Once applied, the coating prevents bacterial and contaminant entry into the wound, thus reducing the rate of secondary infection. Generally, the adhesive coating does not limit dexterity and promotes faster wound healing. Additionally, unlike conventional bandages, the sealant naturally sloughs off the skin within 2-3 days after application and, accordingly, avoids the discomfort associated with removal of conventional bandages from the skin. However, if early removal of this polymeric coating is desired, such can be achieved by use of solvents such as acetone. Further discussion of this use may be found in U.S. Pat. No. 6,342,213.

By way of elaboration it should be noted that several wound care products are currently being marketed which contain an antiseptic benzalkonium chloride and an antibiotic mixture of polymixin B-sulfate and bacitracin-zinc. Patents in this area of technology have described the use of commonly known antiseptics and antibiotics, such as those described in U.S. Pat. Nos. 4,192,299, 4,147,775, 3,419,006, 3,328,259, and 2,510,993. U.S. Pat. No. 6,054,523, to Braun et al., describes materials that are formed from organopolysiloxanes containing groups that are capable of condensation, a condensation catalyst, an organopolysiloxane resin, a compound containing a basic nitrogen, and polyvinyl alcohol. U.S. Pat. No. 5,112,919, reported a moisture-crosslinkable polymer that was produced by blending a thermoplastic base polymer, such as polyethylene, or a copolymer of ethylene, with 1-butene, 1-hexene, 1-octene, or the like; a solid carrier polymer, such as ethylene vinylacetate copolymer (EVA), containing a silane, such as vinyltrimethoxysilane; and a free-radical generator, such as an organic peroxide; and heating the mixture. The copolymers could then be cross-linked by reaction in the presence of water and a catalyst, such as dibutyltin dilaurate, or stannous octoate. U.S. Pat. No. 4,593,071 to Keough reported moisture cross-linkable ethylene copolymers having pendant silane acryloxy groups.

A polyurethane wound coating is described by Tedeshchl et al., in EP 0992 252 A2, where a lubricious, drug-accommodating coating is described that is the product of a polyisocyanate; an amine donor, and/or a hydroxyl donor; and an isocyanatosilane adduct having terminal isocyanate groups and an alkoxy silane. A water soluble polymer, such as poly(ethylene oxide), can optionally be present. Cross-linking causes a polyurethane or a polyurea network to form, depending upon whether the isocyanate reacts with the hydroxyl donors or the amine donors. U.S. Pat. No. 6,967,261 describes the use of chitosan in wound treatment. Chitosan is a deacetylated product of chitin (C₈H₁₃NO₅)ₙ, an abundant natural glucosamine polysaccharide. In particular, chitin is found in the shells of crustaceans, such as crabs, lobsters and shrimp. The compound is also found in the exoskeletons of marine zooplankton, in the wings of certain insects, such as butterflies and ladybugs, and in the cell wall of yeasts, mushrooms and other fungi. Antimicrobial properties of chitosan have been reported against Gram positive and Gram negative bacteria, including Streptococcus spp., Staphylococcus aureus, Staphylococcus epidermidis, Staphylococcus haemolyticus, Pseudomonas, Escherichia, Proteus, Klebsiella, Serratia, Acinobacter, Enterobacter and Citrobacter spp. Chitosan has also been described in the literature to induce repair of tissue containing regularly arranged collagen bundles.

The composition may also be used to close wounds much like stitches or bandages. To be used in such a way, the composition is applied to at least one skin surface of the opposed skin sections of, for example, a suturable wound of a mammalian patient (e.g., human patient). The opposed skin sections are contacted with each either before or after application of the composition. In either case, after application of the composition, the wound area is maintained under conditions wherein the composition polymerizes to join these skin sections together. In general, a sufficient amount of the composition may be employed to cover the wound and the adjacent the skin surface of at least one of the opposed skin sections of the suturable wound. Upon contact with skin moisture and tissue protein, the composition will polymerize or, in the case of compositions utilizing partially polymerized monomers, will further polymerize, at ambient conditions (skin temperature) over about 10 seconds to 60 seconds to provide a solid polymeric film which joins the skin sections, thereby closing the wound. Generally, the composition can provide a polymeric film over the separated skin sections thereby inhibiting infection of the wound while promoting healing. Further discussion of this use may be found in U.S. Pat. No. 6,214,332.

The composition may be packaged in a “kit” form for use in medical facilities and bundled with the appropriate skin prep solution for ease of use and the convenience of the medical personnel. Kits may also include a container holding the skin sealant composition and another separate container for the tannate as previously described. The kit may also include an applicator and means for mixing the contents of the two containers. Alternatively the tannate may be impregnated onto a sponge which is used to apply the sealant and through which the skin sealant flows when it is dispensed. In addition, various complimentary or “mating” containers and different packaging schemes have been used for some time and are known in the art.

The following examples show the efficacy of the instant approach.

### EXAMPLE 1

### Iron Tannate in Skin Prep

Water and alcohol (70% isopropanol) solutions were prepared containing 0.2% wt/wt tannic acid or iron (III) chloride. When these solutions were mixed a deep blue colored solution was generated. When this deeply colored solution was mixed with acid or skin sealant the color was discharged to leave a very pale yellow solution.

Iron tannate solution (0.3% wt/wt) in 70% isopropanol (IPA) was swabbed onto Vitroskin® artificial skin and allowed to dry to yield a blue colored patch. Vitroskin® is available from IMS Inc., of Orange, Conn and is hydrated over glycerol/water for 12 hours before use as described in the product instructions. A sample of InteguSeal® skin sealant was applied to this area and the skin sealant discharged the color in <1 min.

### EXAMPLE 2

### Iron Tannate in the Patient Preoperative Skin Preparation (“Skin Prep”) with the Color Discharged by the “Acidic” Sealant

Iron tannate was dissolved into an alcohol-based chlorhexidine (2% w/v chlorhexidine in isopropanol, 70% w/v) solution (100 mg in 20 ml of solution) to yield a blue-black solution. This skin prep solution was applied to Vitroskin® artificial skin via a sponge application to yield a black colored square 4″×4″ (10.2 cm by 10.2 cm). After allowing for the skin prep to dry, cyanoacrylate skin sealant was applied via the InteguSeal® applicator foam tip. The acidic nature of the skin sealant was sufficient to discharge the black color to yield a colorless and transparent coating.

### EXAMPLE 3

### Tannic Acid (Aldrich Chemical Co., Milwaukee, Wis.) in the Skin Sealant with Iron Chloride in the Foam Applicator

To 10 ml of the InteguSeal® skin sealant was dissolved 500 ppm tannic acid and placed in a glass ampoule. The foam tip of the applicator was dip coated with 3% wt/wt solution of iron chloride in isopropanol and allowed to air dry in a fume hood. The applicator was then assembled by first placing the ampoule containing the tannic acid and sealant into the body of the applicator and then fitting the foam tip onto the end of the applicator. The applicator was then activated and the skin sealant applied to a sheet of Vitroskin® artificial skin. The skin sealant turned blue-black on coming through the foam (due to the formation of iron tannate) and went onto the skin to initially produce a black coating which slowly became pale blue-black on curing. This loss of color was due to the acidic nature of the skin sealant reacting with the iron tannate, resulting in significant discharge the color.

### EXAMPLE 4

### Iron Tannate in the Foam Applicator Tip with Citric Acid in the Alcohol-Based Skin Preparation

The foam applicator tip of the InteguSeal® applicator was soaked in a solution of iron tannate (500 ppm) in isopropanol and then allowed to air dry. The applicator was then reassembled and the foam fitted. To a 10 ml solution of 2% w/v chlorhexidine in isopropanol, 70% w/v skin prep was added 100 mg of citric acid and stirred to dissolve. This solution was then applied to a sheet of Vitroskin® skin in a 6″×6″ (15.2 cm by 15.2 cm) square area using a foam applicator. The InteguSeal® applicator was then activated and sealant applied to the skin prep area. The blue-black skin sealant coated the skin prep area and within 20 seconds the was completely colorless and transparent. The citric acid shortened the time to discharge the color of the sealant containing the iron tannate.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A composition comprising a tannate that changes color when said composition comes in contact with an acid.

2. The composition of claim 1, wherein said tannate is iron tannate.

3. The composition of claim 1, wherein said tannate is ferric tannate.

4. The composition of claims 1 which is applied over a skin preparation.

5. The composition of claim 1, wherein the color change is visible to the human eye under normal light conditions.

6. The composition of claim 1 comprising a cyanoacrylate-based resin.

7. The composition of claim 1 wherein said tannate is present in an amount between about 0.09 and 10 weight percent of the composition.

8. The composition of claim 1 wherein said tannate is impregnated onto a sponge which is used to apply said composition.

9. The composition of claim 1 wherein said tannate is applied separately to a surface from a separate reservoir to produce said composition.

10. The composition of claim 1 used to cover wounds, abrasions, burns, acne, blisters and other disruptions in the skin to protect the skin from subsequent contamination.

11. The composition of claim 10 wherein said skin is human or animal skin.

12. The composition of claim 1 used to close wounds.

13. The composition of claim 1 packaged in a “kit” form.

14. The composition of claim 1 which is bundled with an iodine-containing skin prep solution.

15. A medical kit comprising a skin sealant and a tannate that changes color upon contact with an acid.

16. The medical kit of claim 14 further comprising an iodine-containing skin prep.

17. The medical kit of claim 14 wherein said tannate is impregnated onto a sponge which is used to apply said sealant.
